(19) United States
(12) Patent Application Publication
(10) Pub. No.: US 2003/0232430 A1
(43) Pub. Date: Dec. 18, 2003

(54) Methods for making and using reprogrammed human somatic cell nuclei and autologous and isogenic human stem cells

(75) Inventors: Jose Cibelli, Holden, MA (US); Michael West, Southborough, MA (US); Keith Campbell, Loughborough (GB)

Correspondence Address: CROWELL & MORING, L.L.P., Intellectual Property Group, P.O. Box 14300, Washington, DC, 20044-4300, US

(73) Assignee: Advanced Cell Technology, Worcester, MA

(21) Appl. No.: 10/304,020

(22) Filed: Nov. 26, 2002

Related U.S. Application Data
(60) Provisional application No. 60/332,510, filed on Nov. 26, 2001.

(51) Int. Cl. C12N 5/08

(52) U.S. Cl. 435/366

(57) ABSTRACT

Activated human embryos produced by therapeutic cloning can give rise to human totipotent and pluripotent stem cells from which autologous cells for transplantation therapy are derived. The present invention provides methods for producing activated human embryos that can be used to generate totipotent and pluripotent stem cells from which autologous cells and tissues suitable for transplantation can be derived. In one embodiment, the invention provides methods for producing activated human embryos by parthenogenesis; in another embodiment, the invention provides methods for producing activated human embryos by somatic cell nuclear transfer whereby the genetic material of a differentiated human donor cell is reprogrammed to form a diploid human pronudeus capable of directing a cell to generate the stem cells from which autologous, isogenic cells for transplantation therapy are derived. The ability to create autologous human embryos represents a critical step towards generating immune-compatible stem cells that can be used to overcome the problem of immune rejection in regenerative medicine. The activated human embryos produced by the present invention also provide model systems for identifying and analyzing the molecular mechanisms of epigenetic imprinting and the genetic regulation of embryogenesis and development.

### RELATED APPLICATION

This application claims priority to U.S. provisional application 60/332,510 filed Nov. 26, 2001 incorporated by reference in its entirety.

### FIELD OF THE INVENTION

The present invention relates to the field of therapeutic cloning, the production of activated human embryos from which totipotent and pluripotent stem cells can be generated, and the derivation from these of cells and tissues suitable for transplantation that are autologous to a patient in of such transplant. In particular, the present invention relates to therapeutic cloning of human cells by parthenogenetic activation of a human embryo, and by nuclear transfer into an oocyte to effect the reprogramming of the genetic material of a human somatic cell to form a diploid human pronucleus capable of directing a cell to generate the stem cells from which autologous, isogenic cells for transplantation therapy are derived. The present invention also relates to the fields of study of the molecular mechanisms of epigenetic imprinting and the genetic regulation of embryogenesis and development.

### BACKGROUND OF THE INVENTION

Until recently, it was thought that the differentiation of stem cells into the different somatic cell types of a mammal is associated with irreversible structural changes in chromatin structure and function that commit the differentiating cells to patterns of genetic expression characteristic of particular somatic cell types. The idea that the genome of somatic cells is irreversibly programmed during differentiation was discredited when nuclear transfer (NT)-derived bovine blastocysts were generated using cumulus cells (4). That the nucleus of a differentiated somatic cell could be reprogrammed to a state capable of directing embryogenesis was later confirmed by Wilmut et al. with the cloning of an adult sheep from a quiescent mammary gland-derived cell (5); and by Cibelli et al. with the cloning of an adult bovine from actively dividing fetal fibroblasts (6). Following these pioneering results, protocols for NT using somatic cells have been improved and extended to new mammalian species; however, little is understood of the mechanisms underlying, and the parameters controlling, the process whereby the genetic material (i.e., the genomic DNA and proteins that form chromatin, the nuclear matrix, nucleoplasm, genetic regulatory factors and complexes, etc.) of a differentiated cell is “reprogrammed” by ooplasm to form a diploid pronucleus that is capable of directing the generation of daughter cells that are, or give rise to, totipotent, near totipotent, or pluripotent stem cells.

There presently is great need for new sources of cells and tissues for therapeutic transplant that are histocompatible with the transplant recipients. Transplanted cells or tissue are rejected by the immune system of the transplant recipient unless they are histocompatible with the recipient. Rejection occurs as a result of an adaptive immune response to alloantigens or xenoantigens on the grafted tissue by the transplant recipient. The alloantigens or xenoantigens are typically on “non-self” proteins, i.e., antigenic proteins that are identified as foreign by the immune system of a transplant recipient. The proteins on the surfaces of transplanted tissue that most strongly evoke rejection are the antigenic proteins encoded by the MHC (major histocompatibility complex) genes. In order to match the types of MHC molecules present in the transplant tissue with those of a recipient, assays are performed to identify the MHC types present on the cells of tissue to be transplanted, and on the cells of the transplant recipient. The number of people in need of cell, tissue, and organ transplants is far greater than the available supply of cells, tissues, and organs suitable for transplantation; as a result, it is frequently impossible to obtain a good match between a recipient's MHC proteins those of cells or tissue that are available for transplant. Hence, many transplant recipients must wait for an MHC-matched transplant to become available, or accept a transplant that is not MHC-matched. If the latter is necessary, the transplant recipient must rely on heavier doses of immunosuppressive drugs and face a greater risk of rejection than would be the case if MHC matching had been possible. New sources of histocompatible cells and tissues for therapeutic transplant to non-human mammals in need of such transplant are also needed in veterinary medicine.

Stem cells as a source of cells and tissues for therapy

Embryonic stem (ES) cells are undifferentiated stem cells that are derived from the inner cell mass of a blastocyst embryo. ES cells appear to have unlimited proliferative potential, and are capable of differentiating into all of the specialized cell types of a mammal, including the three embryonic germ layers (endoderm, mesoderm, and ectoderm), and all somatic cell lineages and the germ line. For example, ES cells can be induced to differentiate in vitro into cardiomyocytes (Paquin et al., Proc. Nat. Acad. Sci. (2002) 99:9550-9555), hematopoietic cells (Weiss et al., Hematol. Oncol. Clin. N. Amer. (1997) 11(6):1185-98; also U.S. Pat. No. 6,280,718), insulin-secreting beta cells (Assady et al., Diabetes (2001) 50(8):1691-1697), and neural progenitors capable of differentiating into astrocytes, oligodendrocytes, and mature neurons (Reubinoff et al., Nature Biotechnology (2001) 19:1134-1140; also U.S. Pat. No. 5,851,832). According to data from the Centers for Disease Control and Prevention, as many as 3,000 Americans die every day from diseases that in the future may be treatable with tissues derived from ES cells. In addition to generating functional replacement cells such as cardiomyocytes, neurons, or insulin-producing β cells, ES cells may be able to reconstitute more complex tissues and organs, including blood vessels, myocardial “patches,” kidneys, and even entire hearts (Atala, A. & Lanza, R. P. Methods of Tissue Engineering, Academic Press, San Diego, Calif., 2001).

In order to fully realize the potential benefits of producing cells and tissues for transplant from ES cells and other totipotent, nearly totipotent, or pluripotent stem cells, sources of adequate quantities of such stem cells that are histocompatible with those in need of transplants must be found, and methods for directing the stem cells to differentiate into all of the different cells needed, and means for purifying them for transplant, must be obtained.

Stem cells produced by nuclear transfer cloning

Advanced Cell Technology, Inc. (ACT), the assignee of this application, and other groups have developed methods for transferring the genetic information in the nucleus of a somatic or germ cell from a child or adult into an unfertilized egg cell, and culturing the resulting cell to divide and form a blastocyst embryo having the genotype of the somatic or germ nuclear donor cell. Methods for cloning by such methods, referred to as “somatic cell nuclear transfer” because somatic donor cells are commonly used, are described, for example, in U.S. Pat. Nos. 5,994,619, 6,235,969, and 6,252,133, the contents of which are incorporated herein in their entirety. Totipotent ES or ES-like cells derived from the inner cell mass of a blastocyst generated by somatic cell nuclear transfer have the genomic DNA of the somatic nuclear donor cell, and differentiated cells derived from such ES cells are histocompatible with the individual from whom the somatic donor cell was obtained. Hence, one approach to overcoming the shortage of histocompatible cells and tissues suitable for transplant therapies, is to perform nuclear transfer cloning using a somatic donor cell from the human or non-human mammal that is in need of such a transplant, derive ES cells from the resulting blastocysts, and culture the ES cells under conditions that induce or direct their differentiation into cells of the type that are needed for transplant. Although cloning by nuclear transfer as a means of generating stem cells has been achieved in mice (7-9) and cattle (10), the cloning of primate embryos, including humans, using somatic donor cells has been problematic and has yet to be reported.

Cells and tissues generated by somatic cell nuclear transfer cloning are nearly completely autologous—all of the cells' proteins except those encoded by the cells' mitochondria, which derive from the oocyte, are encoded by the patient's own DNA. Concerns that allogenic mitochondria in cells obtained by somatic cell nuclear transfer cloning and transplanted into a syngeneic transplant recipient would elicit rejection of the transplant have been allayed by recent studies by researchers at ACT showing that cells and tissues produced by nuclear transfer cloning and transplanted into syngenic cattle do not elicit rejection. Tissue-engineered constructs comprising three different differentiated bovine cell types generated by bovine somatic nuclear transplant cloning were transplanted into the syngeneic cattle, where they survived and grew for 12 weeks without rejection, while allogeneic control cells were rejected. See Lanza et al. (Nature Biotechnology, 2002, 20:689-695), the contents of which are incorporated herein in their entirety. Cells and tissues produced by somatic cell nuclear transfer cloning can thus be therapeutically grafted or transplanted to a syngeneic individual without triggering the severe rejection response that results when foreign cells or tissue are transplanted. Recipients of syngeneic cell and tissue transplants produced by somatic cell nuclear transfer cloning therefore do not need to be exposed to the risk of serious and potentially life-threatening complications that are associated with the use of immunosuppressive drugs and/or immunomodulatory protocols to prevent rejection of allogeneic transplants.

Methods that use nuclear transfer cloning to produce cells and tissues for transplant therapies that are histocompatible with the transplant recipient are described in co-owned and co-pending U.S. Application No. 09/797,684 filed Mar. 5, 2001, which further describes assay methods for determining the immune-compatibility of cells and tissues for transplant; U.S. Application No. 10/112,939 filed Apr. 2, 2002, which also describes methods for inducing stem cells to differentiate into cell types useful for transplant therapy; and U.S. Application No. 10/227,282 filed Aug. 26, 2002 with priority to U.S. Provisional Application No. 60/314,316 filed Aug. 24, 2001, which also describes methods for screening to identify conditions inducing stem cells to differentiate into cell types useful for transplant therapy. Such methods are also described in co-owned and co-pending U.S. Application No. 09/995,659 filed Nov. 29, 2001, and International Application No. PCT/US02/22857 filed Jul. 18, 2002, which further describe methods for producing histocompatible cells and tissues for transplant by androgenesis and gynogenesis, and U.S. Application No. 09/520,879 filed Apr. 5, 2000, which further describes methods for producing “rejuvenated” or “hyper-young” cells having increased proliferative potential relative to cells of the donor animal. Such methods are also described in co-owned and co-pending U.S. Application Nos. 10/228,296 and 10/228,316, both filed on Aug. 27, 2002, which further describe methods for making histocompatible cells and tissues for transplant by trans-differentiation and de-differentiation, respectively, of differentiated somatic cells. The disclosures of all of the above-listed applications are incorporated herein by reference in their entirety.

A bank of ES cells with homozygous MHC alleles for cell transplant therapies

As an alternative to using nuclear transfer cloning to produce syngeneic ES cells de novo and inducing these to differentiate into the required cells for every patient that is in need of therapeutic transplant, nuclear transfer cloning can be used to prepare a bank of pre-made ES cell lines, each of which is homozygous for at least one MHC gene. The MHC genes, in the case of humans also referred to as HLA (human leukocyte antigen) genes or alleles, are highly polymorphic, and a bank of different ES cell lines that includes an ES cell line that is homozygous for each of the variants of the MHC alleles present in the human population will include a large number of different ES cell lines. Once a bank of such ES cells having homozygous MHC alleles is produced, it will be possible to provide a patient in need of cell transplant with MHC-matched cells and tissues by selecting and expanding a line of ES cells from the ES cell bank that has MHC allele(s) that match one of those of the patient, and inducing the ES cells to differentiate into the type of cells that the patient requires. Methods for preparing a bank of ES cell lines that are homozygous for the MHC alleles, and for using these to provide MHC-matched cells and tissues for transplantation therapies are described in the co-pending U.S. Patent Application entitled, “A Bank of Nuclear Transfer-Generated Stem Cells for Transplantation Having Homozygous MHC Alleles, and Methods for Making and Using Such a Stem Cell Bank, filed May 24, 2002, the disclosure of which is incorporated herein by reference in its entirety.

Prior to development of the present invention, there were no published reports of somatic cell nuclear transfer using a human nuclear donor cell that resulted in production of a diploid human pronucleus containing genetic material reprogrammed to be capable of directing the generation of daughter cells that are, or can give rise to, totipotent, near totipotent, or pluripotent stem cells. Hence, there is a need for methods for producing a diploid human pronucleus containing genetic material that is reprogrammed to be capable of directing a cell in the generation of such cells, from which autologous, isogenic cells and tissues suitable for transplantation can be derived.

Cells and tissues for transplant from qynogenetic and androgenetic embryos

Histocompatible cells and tissues suitable for transplant to humans can also be generated from gynogenetic or androgenetic embryos that are produced to have the genomic DNA of a female or male transplant recipient. Such embryos are generally nonviable; but are valuable as sources of stem cells capable of generating autologous cells and tissues suitable for transplant, and as model systems for studying the mechanisms of genetic control over embryogenesis, development, and differentiation.

Under certain conditions that may occur spontaneously or by design in vivo or in vitro, oocytes containing genomic DNA of all-male or all-female origin may become activated and produce a zygote or zygote-like cell that can undergo cleavage and subsequent mitotic division. Gynogenesis is broadly defined as the phenomena wherein an oocyte containing all-female DNA becomes activated and produces an embryo. Gynogenesis includes the production of an embryo having all-female genomic DNA by a process in which the oocyte is activated to complete meiosis by a sperm cell that fails to contribute any genetic material to the resulting embryo. Parthenogenesis is a type of gynogenesis in which an oocyte containing all-female genomic DNA is activated to produce an embryo without any interaction with a male gamete. Parthenogenetically activated oocytes may experience aberrations during the completion of meiosis that result in the production of embryos of aberrant genetic constitutions; e.g., embryos that are polyploid or mixoploid. Androgenesis is in many respects the opposite of gynogenesis; it is a phenomenon whereby an oocyte containing genomic DNA exclusively of male origin is produced and activated to develop into an embryo having all-male genomic DNA. Gynogenetic and androgenetic embryos typically stop developing at a fairly early stage in embryogenesis, because the maternal and paternal chromosomes are structurally and functionally different from each other, and both types of chromosomes are generally needed for normal embryonic development to proceed. Gynogenetic and androgenetic embryos, both haploid and diploid, have been generated from non-human oocytes; but prior to the present invention, there were no reports of human parthenogenotes. There is thus a need for new, improved methods for producing human gynogenetic and androgenetic embryos from which can be generated autologous cells and tissues that are suitable for transplantation to humans in need of such transplants.

Imprinting and epigenetic chromosomal modifications

Genes that are present on both the maternal and paternal chromosomes, but which are differentially expressed, depending on whether they are located on the maternal or the paternal chromosome, are referred to as being imprinted. An example of an imprinted gene is the Igf2 gene that is located on the chromosome 7 and encodes insulin-like growth factor 11 (IGFII), a potent embryonic mitogen. The Igf2 gene on the paternal copy of chromosome 7 is actively expressed in embryonic cells, whereas the maternal copy of chromosome 7 is inactive. The differential expression of imprinted genes in embryonic cells is due to epigenetic structural differences between the maternal and paternal chromosomes; i.e., to structural modifications that do not result in differences in the nucleotide sequences of the genes present on the maternal and paternal chromosomes. Patterns of gene expression are also affected by genomic imprinting in cells of adult mammals. Syndromes and diseases in humans associated with genomic imprinting include Prader-Willi syndrome, Angelman syndrome, uniparental isodisomy, Beckwith-Wiedermann syndrome, Wilm's tumor carcinogenesis and von Hippel-Lindau disease. In animals, genomic imprinting has been linked to coat color. For example, the mouse agouti gene confers wild-type coat color, and differential expression of the Aiapy allele correlates with the methylation status of the gene's upstream regulatory sequences. There currently is great interest in identifying how chromosomes contributed to the embryo by male gametes are structurally and functionally different from the chromosomes contributed to female gametes, e.g., in the regulation of differential expression of imprinted genes, and the role these epigenetic differences play in the development of the embryo. Hence, there is a need for methods for producing haploid and diploid androgenetic and gynogenetic human embryos, and embryos in which reprogramming of diploid genetic material introduced by nuclear transfer is proceeding, as such embryos are useful as model systems for studying the epigenetic structural differences between the chromosomes of sperm and egg, and their role in embryogenesis.

### BRIEF DESCRIPTION OF THE FIGURES

FIG. 1. Pronuclear-stage embryos at 12 h. Scale bar=100 μm.

FIG. 2. Pronuclear-stage embryos at 36 h. Scale bar=100 μm.

FIG. 3. A four-cell embryo at 72 h. The nucleus of the embryo was stained with bisbenzimide (Sigma) and visualized under UV light.

Scale bar=50 μm.

FIG. 4. A six-cell embryo at 72 h. The nucleus of the embryo was stained with bisbenzimide (Sigma) and visualized under UV light.

Scale bar=50 μm.

FIG. 5. Pronuclear-stage embryos produced by nuclear transfer using donor nuclei from human dermal fibroblast cells.

FIG. 6. A cleavage-stage embryo generated by a reconstructed oocyte produced by nuclear transfer using a donor nucleus from a human dermal fibroblast.

FIG. 7. MII oocytes at the time of retrieval. Scale bar=100 μm.

FIG. 8. Four- to six-cell embryos 48 hours after parthenogenetic activation. Distinguishable single-nucleated blastomeres (n) were consistently observed. Scale bar=100 μm.

FIG. 9. Blastocoele cavities (arrows) in embryos produced by parthenogenetic activation were detected on day 6 and maintained in culture until day 7. Scale bar=100 μm.

FIG. 10. Human parthenogenetic blastocyst having an inner cell mass.

FIG. 11. Human ES-like cells derived from cultured ICM cells.

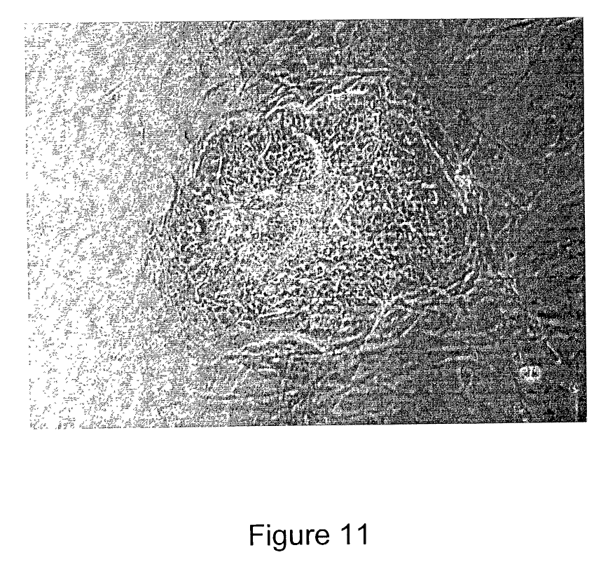

### DETAILED DESCRIPTION OF THE INVENTION

Terms used in the application:

As used herein, a “stem cell” is a cell that has the ability to proliferate in culture, producing some daughter cells that remain relatively undifferentiated, and other daughter cells that give rise to cells of one or more specialized cell types; and “differentiation” refers to a progressive, transforming process whereby a cell acquires the biochemical and morphological properties necessary to perform its specialized functions. Stem cells therefore reside immediately antecedent to the branch points of the developmental tree.

As used herein, an “embryonic stem cell” (ES cell) is a cell line with the characteristics of the murine embryonic stem cells isolated from morulae or blastocyst inner cell masses (as reported by Martin, G., Proc. Natl. Acad. Sci. USA (1981) 78:7634-7638; and Evans, M. and Kaufman, M., Nature (1981) 292: 154-156); i.e., ES cells are capable of proliferating indefinitely and can differentiate into all of the specialized cell types of an organism, including the three embryonic germ layers, all somatic cell lineages, and the germ line.

As used herein, an “embryonic stem-like cell” (ES-like cell) is a cell of a cell line isolated from an animal inner cell mass or epiblast that has a flattened morphology, prominent nucleoli, is immortal, and is capable of differentiating into all somatic cell lineages, but when transferred into another blastocyst typically does not contribute to the germ line. An example is the primate “ES cell” reported by Thomson et al. (Proc. Natl. Acad. Sci. USA. (1995) 92:7844-7848).

As used herein, “inner cell mass-derived cells” (ICM-derived cells) are cells directly derived from isolated ICMs or morulae without passaging them to establish a continuous ES or ES-like cell line. Methods for making and using ICM-derived cells are described in co-owned U.S. Pat. No. 6,235,970, the contents of which are incorporated herein in their entirety.

As used herein, an “embryonic germ cell” (EG cell) is a cell of a line of cells obtained by culturing primordial germ cells in conditions that cause them to proliferate and attain a state of differentiation similar, though not identical to embryonic stem cells. Examples are the murine EG cells reported by Matsui, et al, 1992, Cell 70: 841-847 and Resnick et al, Nature. 359: 550-551. EG cells can differentiate into embryoid bodies in vitro and form teratocarcinomas in vivo (Labosky et al., Development (1994) 120:3197-3204). Immunohistochemical analysis demonstrates that embryoid bodies produced by EG cells contain differentiated cells that are derivatives of all three embryonic germ layers (Shamblott et al., Proc. Nat. Acad. Sci. U.S.A. (1998) 95:13726-13731).

As used herein, a “totipotent” cell is a stem cell with the “total power” to differentiate into any cell type in the body, including the germ line following exposure to stimuli like that normally occurring in development. An example of such a cell is an ES cell, an EG cell, an ICM-derived cell, or a cultured cell from the epiblast of a late-stage blastocyst.

As used herein, a “nearly totipotent cell” is a stem cell with the power to differentiate into most or nearly all cell types in the body following exposure to stimuli like that normally occurring in development. An example of such a cell is an ES-like cell.

As used herein, a “pluripotent cell” is a stem cell that is capable of differentiating into multiple somatic cell types, but not into most or all cell types. This would include by way of example, but not limited to, mesenchymal stem cells that can differentiate into bone, cartilage and muscle; hemotopoietic stem cells that can differentiate into blood, endothelium, and myocardium; neuronal stem cells that can differentiate into neurons and glia; and so on.

Stem cells

The stem cells made by and used for the methods of the present invention may be any appropriate totipotent, nearly totipotent, or pluripotent stem cells. Such cells include inner cell mass (ICM) cells, embryonic stem (ES) cells, embryonic germ (EG) cells, embryos consisting of one or more cells, embryoid body (embryoid) cells, morula-derived cells, as well as multipotent partially differentiated embryonic stem cells taken from later in the embryonic development process, and also adult stem cells, including but not limited to nestin positive neural stem cells, mesenchymal stem cells, hematopoietic stem cells, pancreatic stem cells, marrow stromal stem cells, endothelial progenitor cells (EPCs), bone marrow stem cells, epidermal stem cells, hepatic stem cells and other lineage committed adult progenitor cells.

Totipotent, nearly totipotent, or pluripotent stem cells, and cells therefrom, for use in the present invention can be obtained from any sources of such cells. One means for producing totipotent, nearly totipotent, or pluripotent stem cells, and cells therefrom, for use in the present invention is via nuclear transfer into a suitable recipient cell as described, for example, in co-owned U.S. Pat. No. 5,45,577, and U.S. Pat. No. 6,215,041, the disclosures of which are incorporated herein by reference in their entirety. Nuclear transfer using an adult differentiated cell as a nucleus donor facilitates the recovery of transfected and genetically modified stem cells as starting materials for the present invention, since adult cells are often more readily transfected than embryonic cells. Other aspects of cloning by nuclear transfer leading to production of totipotent, nearly totipotent, or pluripotent stem cells, are also described in the co-owned and co-pending U.S. Patent Applications that are listed above in the section of the application describing the background of the invention, and are also incorporated herein by reference.

Producing autologous cells for transplant

Emerging embryonic stem cell-based technologies offer the potential for many novel therapeutic modalities. However, clinical implementation requires a definitive resolution of the problem of histocompatibility. The ability to generate totipotent stem cells that carry the nuclear genome of the patient using nuclear transfer (NT) techniques would overcome this last major challenge in transplantation medicine (1). It would enable the production of virtually all cell and tissue types, all carrying the nuclear genome of the patient. And since a starting somatic cell can be cultured in vitro without losing its capacity to function as a nuclear donor cell, the starting somatic cell can be genetically modified by gene targeting (2), and the resulting cells produced by using the modified cell as a nuclear donor cell in nuclear transfer would also carry the genetic modification. Clinical applications include the production of cardiomyocytes to replace damaged heart tissue, or insulin producing B-cells for patients with diabetes, among many others (3). However, the implementation of these therapies relies on the generation of early-stage embryos for the purpose of stem cell isolation.

Embryo reconstitution and reprogramming

Embryo reconstitution by nuclear transfer depends upon a number of physical, chemical, and biological variables such as oocyte quality, enucleation and cell transfer procedures, oocyte activation. Successful production of a reconstituted embryo that can undergo cleavage and further development requires that the genetic material of the donor somatic cell be reprogrammed by the oocyte. The mechanism of reprogramming, the nuclear components involved, and the parameters that control it are not understood. Reprogramming is recognized as being a process that affects the function and presumably the structure of the genetic material of the donor nucleus. Nuclear components that may be biochemically modified during reprogramming include the genomic DNA, histone and non-histone chromatin proteins, the nuclear matrix, and soluble proteins and peptides and other nuclear constituents of the nucleoplasm, including regulatory factors that control or modulate the pattern of gene expression (stimulatory and inhibitory transcription factors, complexes, etc.). Reprogramming may include epigenetic structural modifications of the chromatin of the donor nucleus, such as changes in the pattern of DNA methylation and histone acetylation. Reprogramming also appears to be influenced the stage of development and the cell cycle state of the both the nuclear donor cell and the oocyte (6,16-23). The most important effect of reprogramming the donor nucleus appears to be to change the pattern of genetic expression from that of a differentiated cell to a pattern of genetic expression characteristic of an embryonic cell—one that is ultimately capable of directing an embryonic cell to divide mitotically and form daughter cells that are, or give rise to, totipotent, near totipotent, or pluripotent stem cells.

Production of a diploid pronucleus

The present invention is grounded in the discovery that the nucleus of a differentiated human cell can be transferred into a human oocyte such that the genetic material of the differentiated cell forms a diploid pronucleus within the cytoplasm of the oocyte. The transformation of the genetic material of the differentiated cell into a diploid pronucleus is an essential step in the process of reprogramming of the genetic material of the differentiated cell to be capable of directing the generation of daughter cells that are, or give rise to, totipotent, near totipotent, or pluripotent stem cells. The present invention provides methods whereby the nucleus of a differentiated human cell is exposed to ooplasm under conditions such that the nucleus is transformed into a diploid pronucleus. The present invention further provides methods whereby the genetic material in the nucleus of a differentiated human cell is exposed to ooplasm under conditions such that the genetic material is reprogrammed to be capable of directing the generation of daughter cells that are, or can give rise to, totipotent, near totipotent, or pluripotent stem cells. Natural pronuclei that result from the remodeling of the oocyte and sperm nuclei after fertilization are haploid, and their fusion during syngamy does not result in formation of a single diploid pronucleus. Diploid human pronuclei produced by the present invention do not occur naturally, and would not exist but for the hand of Man.

One embodiment of the present invention comprises transferring the nucleus of a differentiated human cell into a human oocyte, while at approximately the same time, removing the endogenous chromosomes from the recipient oocyte. As a result of being exposed to the cytoplasm of the oocyte, the genetic material of the transferrred nucleus becomes transformed into a diploid pronucleus.

The diploid pronucleus produced by exposure to ooplasm can be used to direct embryonic development to generate isogenic cells that are suitable for transplantation therapy. Foe example, a diploid pronucleus produced by the present invention can be left within the reconstituted oocyte so that the genetic material is reprogrammed to direct embryonic development when it becomes genetically active (at around the 8 cell stage). When the embryo develops into the blastocyst having an inner cell mass (ICM), the ICM cells can be isolated and cultured to generate embryonic stem (ES) cells, as described below. Human ES cells produced in this manner can be induced to form pluripotent stem cells and differentiated cell types that are suitable for transplantation therapy.

Alternatively, a diploid pronucleus produced by the present invention can be extracted from the reconstituted oocyte and transferred into another enucleated oocyte, or into an enucleated fertilized zygote, where it can direct embryonic development upon becoming genetically active. Examples of such a double nuclear transfer method are described in International Application No. PCT/GB00/00086 of Campbell, and in Heindryckx et al. (Biol. Reprod., 2002, 67(6):1790-5), the contents of both of which are incorporated herein by reference in their entirety. Methods for extacting and transferring pronuclei for such methods are well known; for example, see Liu et al. (Hum. Reprod., 2000, 15(9):1997-2002) and Ivakhenko et al. (Hum. Reprod., 2000, 15(4):911-6), the contents of both of which are incorporated herein by reference in their entirety.

Early human reconstituted embryos, including 2-cell, 4-cell, 8-cell, morula, and blastocyst embryos, produced by the present invention, can be dissaggregated by known methods, and the one or more of the embryonic cells can be inserted into an evacuated zona, where the cell or cells will proceed to develop into embryos that can be used to generate generate isogenic cells suitable for transplantation therapy. Examples wherein such methods are used to produce multiple, identical embryos are described in Johnson et al., (Vet. Record, 1995, 137:15-16), Willadsen (J. Reprod. Fert., 1980, 59:357-62), and Willadsen (Vet. Record, 1981, 108:211-3); the contents of which are incorporated herein by reference in their entirety. It is recognized by persons skilled in the art that the greater the number of embryos cultured to produce ICM cells that give rise to ES, the greater the probability that such ES cells will be obtained.

Early human reconstituted embryos, including 2-cell, 4-cell, 8-cell, morula, and blastocyst embryos, produced by the present invention, can also be dissaggregated by known methods, and individual embryonic cells can be used as nuclear donor cells and fused with enucleated oocytes using known methods of cloning by nuclear transfer, for production of embryos that can be used to generate generate isogenic cells suitable for transplantation therapy. Examples wherein such methods are used to produce multiple, identical embryos are described in Takano et al. (Theriogenology, 1997, 147:1365-73), and Lavoir et al. (Biol. Reprod., 1997, 56:194-199), the contents of which are incorporated herein by reference in their entirety.

The present invention also includes methods for producing a diploid pronucleus comprising exposing the nucleus or genetic material of a differentiated human cell to ooplasm by means other than nuclear transfer into a human oocyte. For example, ooplasm can be introduced into a differentiated human cell by fusing the cell with blebs containing oocyte cytoplasm as described in co-owned and co-pending U.S. Application No. 09/736,268 of Chapman, the contents of which are incorporated herein by reference in their entirety. Ooplasm can also be introduced into a differentiated human cell by electroporation as described in co-owned and co-pending U.S. Application No. 10/228,316 of Dominko et al., the contents of which are incorporated herein by reference in their entirety.

A human diploid pronucleus can also be produced by exposing the nucleus or genetic material of a differentiated human cell to ooplasm of a non-human oocyte; e.g., by nuclear transfer, for example, as described in co-owned and co-pending U.S. Application No. 09/685,061 of Robl et al., the contents of which are incorporated herein by reference in their entirety.

Embryonic cells formed by cleavage of a reconstituted embryo formed according to the present invention are also useful in performing karyotype analysis. See Verlinskey et al. (Fertil. Steril., 1999, 72(6):1127-33), the contents of which are incorporated herein by reference in their entirety.

Reprogramming nuclei of differentiated human cells:

The following set of procedures is presented to describe steps of the embodiment of the invention wherein a human diploid pronucleus is generated by transferring the nucleus of a differentiated human cell into a human oocyte. These procedures comprise using human nuclear transfer to produce a human diploid pronucleus, to effect the reprogramming of the genetic material of a differentiated somatic cell, and to generating embryonic cells that can give rise to totipotent, near totipotent, and pluripotent cells. Persons skilled in the art would appreciate that the values of the parameters of the various steps of the methods described below can be varied and reagents used in the methods can be substituted by different reagents having similar properties without substantially altering the character of the procedures or their results, or departing from the invention disclosed herein.

A. Collecting oocytes—the oocytes obtained by this method can be used either for reprogramming somatic cell nuclei by nuclear transfer, or for parthenogenetic activation:

1 Oocytes are aspirated from follicles by known procedures at 30 to 50 hrs post hCG administration; e.g., by using an ultrasound-guided needle.

2 Oocytes are denuded of cumulus cells by known procedures; e.g., by pipetting up and down using a finely pulled pipette in suitable media containing hyaluronidase (e.g., 1 mg/ml hyaluronidase in Hanks media).

3 Denuded oocytes are placed in suitable medium, such as Hanks with 1% Bovine Serum Albumin (BSA) or Hanks with 1% Human Serum Albumin (HSA), and are transported to the laboratory where the parthenogenetic activation or nuclear transfer procedure is to be performed.

4 Within zero to about 12 hours after recovery, the oocytes are placed in a drop of G1 (SERIES III), or KSOM, or GEM with suitable cell culture medium under mineral oil, and are incubated until parthenogenetic activation or nuclear transfer is performed. For example, good results are obtained by placing oocytes in a drop of 500 μl of G1 (SERIES III), or KSOM, or GEM, with 5 mg/ml HSA culture media under mineral oil, and incubating at 37°C. in 6% CO₂ in air until parthenogenetic activation or nuclear transfer is performed.

A. Somatic cell preparation:

1 An in vitro culture of differentiated somatic donor cells is dissociated and suspended using a solution of trypsin-EDTA in calcium-free Dulbecco's phosphate buffered saline (DPBS, Sigma); e.g., for five minutes at room temperature. Once a suspension of single cells is obtained, the enzymatic activity is neutralized; for example, by adding 30% fetal calf serum.

2 The cell suspension is spun gently to pellet the cells; e.g., at 500 g for 10 minutes.

3 The supernatant is discarded and the cell pellet is re-suspended in suitable medium; e.g., in Human Tubule Fluid (HTF) containing 1 mg/ml of HSA. The cells can be used as donor cells for nuclear transfer within 0 to 24 hours after dissociation.

Alternatively—

Cells to be used as nuclear donor cells (e.g. white blood cells or granulosa/cumulus cells from the oocytes) are taken directly from the human donor and are placed in suitable medium; e.g., in HTF containing 1 mg/ml of HSA. The cells can be used as donor cells for nuclear transfer within 0 to 5 days after isolation.

B. Nuclear Transfer

1 Oocytes are taken from the drop of G1 (SERIES III) or KSOM or GEM+culture medium under mineral oil, and are moved to a drop of G1 (SERIES III) or KSOM or GEM+culture medium containing 33342 Hoechst and are incubated for about 6 to 18 minutes to label the oocyte chromatin. For example, the oocytes can be moved to a 500 μl drop of G1 (SERIES III), or KSOM, or GEM, with 5 mg/ml HSA culture media containing 1 μg/ml 33342 Hoechst dye under mineral oil, and incubated for 15 minutes at 37° C. in 6% CO₂ in air.

2 Somatic donor cells are placed into a manipulation drop of 100 μl of HTF containing 1 mg/ml HSA, 20% FCS, and 10 μg/ml cytochalasin B under mineral oil.

3 Oocytes are moved into a manipulation drop of 100 μl of HTF containing 1 mg/ml of HSA, 20% FCS and 10 μg/ml cytochalasin B under mineral oil adjacent to the drop containing the somatic donor cells, and the whole plate (e.g., a 100 mm Falcon plate) is placed at 37° C. in the warming stage of the microscope.

4 After about 15 minutes—

a. The metaphase 11 plate (of chromosomes) in the oocyte is visualized under ultraviolet light for no more than 5 seconds, and a laser (______) is used to drill a 20 micron hole in the zona pellucida adjacent to the MII plate.

b. Chromosomes at the MII plate are suctioned into a fire-polished glass pipette with an inner diameter (I.D.) of 20 μm without compromising the integrity of the oocyte.

c. One small somatic donor cell is picked up using a fire-polished 20 μm I.D. glass pipette and is placed in the perivitelline space of the oocyte.

Alternatively, instead of piercing the zona pellucida with a laser—

A beveled pipette is used to pierce the zona pellucida; or

A pipette filled with tyroid acid is used to drill the zona similar to the procedure used during assisted hatching; or

A Piezo electric device (Prime Tech) is used to drive a blunt glass pipette to a point immediately adjacent to the MII plate.

5 Couplets (oocyte and somatic cell) produced by the above-described procedure are moved from the manipulation drop into a drop of 500 μl of G1 (SERIES III), or KSOM, or GEM, with 5 μg/ml HSA culture medium under mineral oil, and are incubated at 37° C. in 6% CO₂ until fusion is performed.

6 At 0 to about 24 hours after cell transfer, the oocytes are moved out of the drop of G1 (SERIES III), or KSOM, GEM,+culture medium under mineral oil and into a cell culture plate (e.g., a 30 mm Falcon plate) containing 3 ml of HTF with 1 mg/ml of HSA, and are incubated for 30 seconds.

7 The couplets are then moved into a solution of 50% HTF with 1 mg/ml HSA and 50% fusion media (Sorbitol based) for 1 minute.

8 Couplets are moved to a solution of 100% fusion media

9 Couplets are moved to a BTX fusion chamber (500 μl gap) filled with fusion media and placed between two electrodes.

10 Alignment of the couplets is performed manually using a glass pipette in a way that the axis of the somatic cell and oocyte is perpendicular to the axis of the electrodes.

11 One to ten fusion pulses of 150 volts for 15 μseconds are delivered.

12 Couplets are immediately moved into a solution of 50% HTF with 1 mg/ml HSA and 50% fusion media (Sorbitol or Manitol or Glucose based) for 1 minute.

13 Couplets are moved into a cell culture plate (e.g., a 30 mm Falcon plate) containing 3 ml of HTF with 1 mg/ml of HSA for 1 minute.

14 Couplets are then moved into a drop of 500 μl of G1 (SERIES III), or KSOM, or GEM, with 5 mg/ml HSA culture media under mineral oil, and are incubated at 37° C. in 6% CO₂ in air until activation is performed.

Alternatively—

A Piezo electric device (Prime Tech) is used to drive a blunt glass pipette that injects the nucleus of the somatic cell.

C. Oocyte Activation

1 At somewhere between 30 to 50 hours after hCG administration, fused reconstructed embryos are placed into a solution of 10 μM of ionomycin in HTF with 1 mg/ml of HSA for 1 to 20 minutes.

2 Reconstructed embryos are moved into a drop of 500 μl of a solution of 2 mM 6-DMAP in G1 (SERIES III), or KSOM, or GEM, with 5 mg/ml HSA culture media under mineral oil, and are incubated at 37° C. in 6% CO₂ in air for 0.5 to 24 hours.

3 Reconstructed embryos are taken out of DMAP solution and rinsed three times in three different (30 mm Falcon) plates of HTF with 1 mg/ml HSA.

4 Reconstructed embryos are moved into a drop of 500 μl of G1 (SERIES III), or KSOM, OR GEM, with 5 mg/ml HSA culture media under mineral oil, and are incubated at 37° C. in 6% CO₂ in air.

D. Embryo Culture

1 For the first 72 hours, the reconstructed embryos are cultured in a drop of 500 μl of G1 (SERIES III), or KSOM, or GEM, with 5 mg/ml HSA culture media under mineral oil, and are incubated at 37° C. in 6% CO₂ in air.

2 For the rest of the culture period (from hour 73 until blastocyst), the embryos are cultured in a drop of 500 μl of KSOM+AA+Glucose (Specialty media) with 5 mg/ml HSA and 10% heat inactivated follicular fluid obtained from superovulated human oocyte donors, under mineral oil, at 37° C. in 6% CO₂ in air.

3 Once blastocysts are generated, inner cell mass (ICM) isolation is performed.

E. Inner Cell Mass Isolation

1 Hatched blastocysts are placed in tyroid acid for a few seconds until the zona pellucida is digested, and then are moved to HTF with 1 mg/ml of HSA for up to 2 minutes.

2 The blastocysts are then moved to solution of polyclonal antibodies (1:5) of serum against BeWo cells in G1 (SERIES III), or KSOM, or GEM, without HSA, for one hour.

3 Embryos are rinsed 3 times in HTF with 1 mg/ml of HAS, and are moved to a solution of guinea pig complement (1:3) in G1 (SERIES III), or KSOM, or GEM, without HAS, until trophoblast lysis occurs.

4 The ICM is rinsed in HTF with 1 mg/ml of HAS, and is placed on a suitable feeder cell layer; e.g., mitotically inactivated mouse embryonic fibroblasts, in DMEM with 15% fetal calf serum.

It is known that artificial activation of mammalian oocytes, including oocytes containing DNA of all male or female origin, can be induced by a wide variety of physical and chemical stimuli. Examples of such methods are listed in the Table below.

List of physical and chemical stimuli which can
induce oocyte activation in mammals.

| Physical | | Chemical | | |
|---|---|---|---|---|
| 1. | Mechanical | 1. | Enzymatic | |
| | (a) pricking | | | trypsin, pronase, hyaluronidase |
| | (b) manipulation of oocytes in vitro | 2. | Osmotic | |
| 2. | Thermal | 3. | Ionic | |
| | (a) cooling | | (a) | divalent cations |
| | (b) heating | | (b) | calcium ionophores |

| | | | |
|---|---|---|---|
| 3. | Electric | 4. | Anaesthetics |

| | |
|---|---|
| (a) | general - ether, ethanol, nembutal, |
| | chloroform, avertin |
| (b) | local - dibucaine, tetracaine, |
| | lignocaine, procaine |

| | |
|---|---|
| 5. | Phenothiazine, tranquillizers |

thioridazine, trifluoperazine,
fluphenazine, chlorpromazine

| | |
|---|---|
| 6. | Protein synthesis inhibitors |

cycloheximide, puromycin

| 7. | Phosphorylation inhibitors (e.g., |
|---|---|
| DMAP) | |
| 8. | Inisitol 1,4,5-triphosphate (Ins P₃) |

Using nuclear transfer procedures similar to those described above, nuclei of two different types of human differentiated somatic cells, fibroblasts and cumulus cells, have been transferred into enucleated human oocytes, resulting in formation of diploid pronuclei and reprogramming of the genetic material of the transferred nuclei into that of dividing embryonic cells. These results, and the methods used to obtain them, are described in more detail in the Examples below.

Therapeutic applications:

Prior to undertaking the studies that led to the development of the present invention, the applicants consulted an ethics advisory board—a panel of independent ethicists, lawyers, fertility specialists and counselors assembled to guide the research efforts of the assignee, Advanced Cell Technology, on an ongoing basis. The ethics board considered five key issues before recommending that the work proceed (See Cibelli et al., Scientific American, Nov. 24, 2001, pp. 45-51)

Therapeutic cloning is distinct from reproductive cloning, which aims to implant a cloned embryo into a woman's uterus leading to the birth of a cloned baby. The inventors of the present invention believe that reproductive cloning has potential risks to both mother and fetus that make it unwarranted at this time, and support a restriction on cloning for reproductive purposes until the safety and ethical issues surrounding it are resolved. Unlike reproductive cloning, which aims to produce an entire organism, human therapeutic cloning does not seek to take development beyond the earliest preimplantation stage.

The goal of therapeutic cloning is to use the genetic material from a patient's own cells to generate autologous cells and tissues that can be transplanted back to the patient. Using therapeutic cloning, it is possible to derive primordial stem cells in vitro, such as embryonic stem cells from the inner cell masses of blastocysts, as a source of cells for regenerative therapy (3). Because the transplanted cells generated by therapeutic cloning are isogenic, they will match the patient's HLA type, and immunorejection of the transplanted cells will be attenuated, if it occurs at all. Animal studies suggest that the totipotent, near totipotent, and pluripotent stem cells produced by the therapeutic cloning methods of the present invention can play an important role in treating a wide range of human disease conditions, including diabetes, arthritis, AIDS, strokes, cancer, and neurodegenerative disorders such as Parkinson's and Alzheimer's disease (24-27). For example, stem cells produced by the disclosed therapeutic cloning techniques can be used to generate pancreatic islets to treat diabetes, or nerve cells to repair damaged spinal cords. In addition to generating individual or small groups of replacement cells, it is likely that the cells produced by the methods disclosed herein can also be used to reconstitute more complex tissues and organs, including blood vessels, myocardial “patches,” kidneys, and even entire hearts (28,29).

The techniques disclosed herein have the potential to reduce or eliminate the immune responses associated with the transplantation of these various tissues, and thus the requirement for immunosuppressive drugs and/or immunomodulatory protocols that carry the risk of serious and potentially life-threatening complications for so many patients that are forced to accept transplant of non-histocompatible cells and tissues, because histocompatible transplants cannot be found.

A recent study shows that allogeneic stem cells produce antigenic cell surface proteins that trigger immunorejection; thus, there is a serious need for the isogenic, autologous cells suitable for therapeutic transplant that can be supplied by the methods of the present invention.

Cells suitable for therapeutic transplant that are produced by the methods of the present invention are syngeneic with cells of the transplant recipient, and so are HLA-matched. Therefore, with respect to the major surface protein determinants of self/non-self that trigger graft rejection, the cells for transplant produced by the present invention are histocompatible with the transplant recipient. A recent study shows demonstrates that cloned cells produced by nuclear transfer may not elicit immunorejection in an isogenic transplant recipient, despite the fact that the cells have mitochondria from a different animal. See Lanza et al. (Nat. Biotech., 2002, 20:689-695). Similar studies being performed with primates (cynomologous monkeys). There remains the possibility that an autologous and/or isogenic transplant produced according to the claimed invention will be rejected, due to antigens encoded by the allogenic mitochondria in cells produced by nuclear transfer, or antigens resulting from genetic recombination in cells produced by parthenogenesis. Nonetheless, immunorejection responses that are elicited by such antigens are expected to be significantly weaker than those elicited by allografts, due to the HLA match between the autologous cells produced by the present invention and those of the autologous or isogenic recipient.

Cells and tissues from embryos produced by nuclear transfer cloning.

In one embodiment of the present invention, cells having significant therapeutic potential for use in cell therapy are derived from early stage embryos that are produced by nuclear transfer cloning. This is a cloning method that comprises transferring a donor cell, or the nucleus or chromosomes of such a cell, into an oocyte, and coordinately removing the oocyte genomic DNA, to produce an embryo from which cells or tissues suitable for transplant can be derived, as described, for example, in co-owned and co-pending U.S. Application Nos. 09/655,815 filed Sep. 6, 2000, and 09/797,684 filed Mar. 5, 2001, the disclosures of which are incorporated herein by reference in their entirety.

To provide histocompatible cells and tissues suitable for transplant, nuclear transfer cloning is carried out using a germ or somatic donor cell from the human or non-human mammal that is the transplant recipient, as described in the aforementioned co-pending U.S. applications. Alternatively, cells and tissues suitable for transplant may be obtained by performing nuclear transfer cloning with a donor cell having DNA comprising MHC alleles that match those of the transplant recipient. Cells and tissues derived from an embryo produced by such a method are not syngenic with, but have the same MHC antigens as the cells of the transplant recipient, so that rejection by the recipient is muted, as described in the co-pending application, “A Bank of Nuclear Transfer-Generated Stem Cells for Transplantation Having Homozygous MHC Alleles, and Methods for Making and Using Such a Stem Cell Bank, filed May 24, 2002, the disclosure of which is incorporated herein by reference in its entirety.

The present invention makes it possible to offer therapeutic cloning or cell therapy arising from parthenogenesis to patients in need of transplantation therapy. Currently, efforts are focused on diseases of the nervous and cardiovascular systems and on diabetes, autoimmune disorders, and diseases involving the blood and bone marrow.

Once techniques for deriving nerve cells from cloned embryos are perfected, the inventors expect not only to be able to heal damaged spinal cords but to treat brain disorders such as Parkinson's disease, in which the death of brain cells that make a substance called dopamine leads to uncontrollable tremors and paralysis. Alzheimer's disease, stroke and epilepsy might also yield to such an approach.

Besides insulin-producing pancreatic islet cells for treating diabetes, stem cells from cloned embryos could also be nudged to become heart muscle cells as therapies for congestive heart failure, arrhythmias and cardiac tissue scarred by heart attacks.

A potentially even more interesting application could involve prompting cloned stem cells to differentiate into cells of the blood and bone marrow. Autoimmune disorders such as multiple sclerosis and rheumatoid arthritis arise when white blood cells of the immune system, which arise from the bone marrow, attack the body's own tissues. Preliminary studies have shown that cancer patients who also had autoimmune diseases gained relief from autoimmune symptoms after they received bone marrow transplants to replace their own marrow that had been killed by high-dose chemotherapy to treat the cancer. Infusions of blood-forming, or hematopoietic, cloned stem cells might “reboot” the immune systems of people with autoimmune diseases.

As described in the above-identified patents and co-pending applications, the somatic donor cell used for nuclear transfer to produce a nuclear transplant embryo according to the present invention can be of any germ cell or somatic cell type in the body. For example, the donor cell can be a germ cell or a somatic cell selected from the group consisting of fibroblasts, B cells, T cells, dendritic cells, keratinocytes, adipose cells, epithelial cells, epidermal cells, chondrocytes, cumulus cells, neural cells, glial cells, astrocytes, cardiac cells, esophageal cells, muscle cells, melanocytes, hematopoietic cells, macrophages, monocytes, and mononuclear cells. The donor cell can be obtained from any organ or tissue in the body; for example, it can be a cell from an organ selected from the group consisting of liver, stomach, intestines, lung, stomach, intestines, lung, pancreas, cornea, skin, gallbladder, ovary, testes, kidneys, heart, bladder, and urethra.

As used herein, enucleation refers removal of the genomic DNA from an cell, e.g., from a recipient oocyte. Enucleation therefore includes removal of genomic DNA that is not surrounded by a nuclear membrane, e.g., removal of chromosomes at a metaphase plate. As described in the above-identified patents and co-pending applications, the recipient cell can be enucleated by any of the known means either before, concomitant with, or after nuclear transfer. For example, a recipient oocyte may be enucleated when the oocyte is arrested at metaphase II, when oocyte meiosis has progressed to telophase, or when meiosis has completed and the maternal pronucleus has formed.

As described in the above-identified patents and co-pending applications, the donor genome may be introduced into the recipient cell by injection or fusion of the nuclear donor cell and the recipient cell, e.g., by electrofusion or by Sendai virus-mediated fusion. Suitable testing and microinjection methods are well known and are the subject of numerous issued patents. The donor cell, nucleus, or chromosomes can be from a proliferative cell (e.g., in the G1, G2, S or M cell cycle stage); alternatively, they may be derived from a quiescent cell (in G0).

As described in the above-identified patents and co-pending applications, the recipient cell may be activated prior to, simultaneous with, and/or after nuclear transfer.

Direct harvest of therapeutic cells and tissue from an embryo

Cells or tissue for transplant can be obtained from a nuclear transfer embryo that has been cultured in vitro to form a gastrulating embryo of from about one cell to about 6 weeks of development. For example, cells or tissue for transplant may be obtained from an embryo of from 15 days to about four-weeks old. Alternatively, in the case of non-human NT embryos, cells or tissue for transplant may be obtained from a gastrulating embryo of up to six weeks old, or older, by transferring an NT embryo into a suitable maternal recipient and allowing it to develop in utero for up to six weeks, or longer. Thereupon, it may be harvested from the uterus of the maternal recipient and used as a source of cells or tissues for transplant.

The therapeutic cells that are obtained from a gastrulating embryo at a developmental stage of from one cell to up to six weeks of age can be pluripotent stem cells and/or cells that have commenced becoming committed to a particular cell lineage, e.g., hepotocytes, myocardiocytes, pancreatic cells, hemagioblasts, hematopoietic progenitors, CNS progenitors and others.

Generation of therapeutic cells and tissue from pluripotent embryonic stem cells

In addition to obtaining cells and tissue for transfer from a gastrulating embryo as described above, cells and tissues for therapeutic transfer according to the invention can be generated from pluripotent and/or totipotent stem cells derived from a nuclear transfer embryo produced by the methods of the invention. As described in co-pending U.S. Application Nos. 09/655,815 and 09/797,684, the disclosures of which are incorporated herein by reference, pluripotent and totipotent stem cells produced by nuclear transfer methods according to the present invention can be cultured using methods and conditions known in the art to generate cell lineages that differentiate into specific, recognized cell types, including germ cells. These methods comprise:

a) inserting a donor cell, or the nucleus or chromosomes of such a cell, into an oocyte or other suitable recipient cell, and coordinately removing the genomic DNA of the oocyte or other recipient cell to produce a nuclear transfer embryo; and

b) generating stem cells and/or differentiated cells or tissue needed for transplant from said embryo having the genomic DNA of the donor cell.

Such a method can be used to generate generate pluripotent stem cells and/or totipotent embryonic stem (ES) cells. Pluripotent stem cells produced in this manner can be cultured to generate cell lineages that differentiate into specific, recognized cell types. The totipotent ES cells produced by nuclear transfer have the capacity to differentiate into every cell type of the body, including the germ cells. For example, the pluripotent and/or totipotent stem cells derived from a nuclear transfer embryo can differentiate into cells selected from the group consisting of immune cells, neurons, skeletal myoblasts, smooth muscle cells, cardiac muscle cells, skin cells, pancreatic islet cells, hematopoietic cells, kidney cells, and hepatocytes suitable for transplant according to the present invention. Because the pluripotent and totipotent stem cells produced by such methods have the patient's own genomic DNA, the differentiated cells and tissues generated from these stem cells are nearly completely autologous—all of the cells' proteins except those encoded by the cells' mitochondria, which derive from the oocyte, are encoded by the patient's own DNA. Accordingly, differentiated cells and tissues generated from the stem cells produced by such nuclear transfer methods can be used for transplantation without triggering the severe rejection response that results when foreign cells or tissue are transplanted.

In preparing the pluripotent and totipotent stem cells having primate genomic DNA according to the present invention, one can employ the methods described in James A. Thomson's U.S. Pat. No. 6,200,806, “Primate Embryonic Cells,” issued Mar. 13, 2001. For example, the Thomson patent describes a method for preparing human pluripotent stem cells comprising:

a) isolating a human blastocyst;

b) isolating cells from the inner cell mass of the blastocyst;

c) plating the inner cell mass cells on embryonic fibroblasts so that inner-cell mass-derived cell masses are formed;

d) dissociating the mass into dissociated cells;

e) replating the dissociated cells on embryonic feeder cells;

f) selecting colonies with compact morphologies and cells with high nucleus to cytoplasm ratios and prominent nucleoli; and

g) culturing the selected cells to generate a pluripotent human embryonic stem cell line.

The disclosure of Thomson's U.S. Pat. No. 6,200,806 is incorporated herein by reference in its entirety. A method for inducing the differentiation of pluripotent human embryonic stem cells into hematopoietic cells useful for transplant according to the present invention is described in U.S. Pat. No. 6,280,718, “Hematopoietic Differentiation of Human Pluripotent Embryonic Stem Cells,” issued to Kaufman et al. on Aug. 28, 2001, the disclosure of which is incorporated herein by reference in its entirety. The method disclosed in the patent of Kaufman et al. comprises exposing a culture of pluripotent human embryonic stem cells to mammalian hematopoietic stromal cells to induce differentiation of at least some of the stem cells to form hematopoietic cells that form hematopoietic cell colony forming units when placed in methylcellulose culture.

Generation of “hyper-young” cells and tissue for transplant

Nuclear transfer cloning methods can also be employed to generate “hyper-young” embryos from which cells or tissues suitable for transplant can be derived. Methods for generating rejuvenated, “hyper-youthful” stem cells and differentiated somatic cells having the genomic DNA of a somatic donor cell of a human or non-human mammal are described in co-owned and co-pending U.S. Application Nos. 09/527,026 filed Mar. 16, 2000, 09/520,879 filed Apr. 5, 2000, and 09/656,173 filed Sep. 6, 2000, the disclosures of which have been incorporated herein by reference in their entirety. For example, rejuvenated, “hyper-youthful” cells having the genomic DNA of a human or non-human mammalian somatic cell donor can be produced by a method comprising:

a) isolating normal, somatic cells from a human or non-human mammalian donor, and passaging or otherwise inducing the cells into a state of checkpoint-arrest, senescence, or near-senescence,

b) transferring such a donor cell, the nucleus of said cell, or chromosomes of said cell, into a recipient oocyte, and coordinately removing the oocyte genomic DNA from the oocyte, to generate an embryo; and

c) obtaining rejuvenated cells from said embryo having the genomic DNA of the donor cell.

The rejuvenated cells obtained from the embryo can be pluripotent stem cells or partially or terminally differentiated somatic cells. As described in the above-identified co-pending applications, rejuvenated pluripotent and/or totipotent stem cells can be generated from a nuclear transfer embryo by a method comprising obtaining a blastocyst, an embryonic disc cell, inner cell mass cell, or a teratoma cell using said embryo, and generating the pluripotent and/or totipotent stem cells from said blastocyst, inner cell mass cell, embryonic disc cell, or teratoma cell.

As described in the above-identified co-pending applications, rejuvenated cells derived from a nuclear transfer embryo according to the present invention are distinguished in having telomeres and proliferative life-spans that that are as long as or longer than those of age-matched control cells of the same type and species that are not generated by nuclear transfer techniques. In addition, the nucleotide sequences of the tandem (TTAGGG)ₙ repeats that comprise the telomeres of such rejuvenated cells are more uniform and regular; i.e., have significantly fewer non-telomeric nucleotide sequences, than are present in the telomeres of age-matched control cells of the same type and species that are not generated by nuclear transfer. Such rejuvenated cells are also have patterns of gene expression that are characteristic of youthful cells; for example, activities of EPC-1 and telomerase in such rejuvenated cells are typically greater than EPC-1 and telomerase activities in age-matched control cells of the same type and species that are not generated by nuclear transfer techniques. Moreover, the immune systems of cloned animals produced by nuclear transfer procedures are shown to be enhanced, i.e., to have greater immune responsiveness, than those of animals that are not generated by nuclear transfer techniques. When introduced into a subject, e.g., a human or non-human mammal in need of cell therapy, the cells and tissues derived from such “hyper-young” embryos are capable of efficiently infiltrating and proliferating at a desired target site, e.g., heart, brain, liver, bone marrow, kidney or other organ that requires cell therapy. Hematopoietic progenitor cells derived from such “hyper-young” embryos are expected to infiltrate into a subject and rejuvenate the immune system of the individual by migrating to the immune system, ie., blood and bone marrow. Similarly, CNS progenitor cells derived from such “hyper-young” embryos are expected to preferentially migrate to the brain, e.g., that of a Parkinson's, Alzheimer's, ALS, or a patient suffering from age-related senility.

Parthenogenetic activation of human oocytes:

The inventors also sought to determine whether it was possible to induce human eggs to divide into early embryos without being fertilized by a sperm or being enucleated and injected with a donor cell. Although mature eggs and sperm normally have only half the genetic material of a typical body cell, to prevent an embryo from having a double set of genes following conception, eggs halve their genetic complement relatively late in their maturation cycle. If activated before that stage, they still retain a full set of genes.

Stem cells derived from such parthenogenetically activated cells would be unlikely to be rejected after transplantation because they would be very similar to a patient's own cells and would not produce many molecules that would be unfamiliar to the person's immune system. (They would not be identical to the individual's cells because of the gene shuffling that always occurs during the formation of eggs and sperm.) Such cells might also raise fewer moral dilemmas for some people than would stem cells derived from cloned early embryos.

Under one scenario, a woman with heart disease might have her own eggs collected and activated in the laboratory to yield blastocysts. Scientists could then use combinations of growth factors to coax stem cells isolated from the blastocysts to become cardiac muscle cells growing in laboratory dishes that could be implanted back into the woman to patch a diseased area of the heart. Using a similar technique, called androgenesis, to create stem cells to treat a man would be trickier. But it might involve transferring two nuclei from the man's sperm into a contributed egg that had been stripped of its nucleus.

Researchers have previously reported prompting eggs from mice and rabbits to divide into embryos by exposing them to different chemicals or physical stimuli such as an electrical shock. As early as 1983, Elizabeth J. Robertson, who is now at Harvard University, demonstrated that stem cells isolated from parthenogenetic mouse embryos could form a variety of tissues, including nerve and muscle. Previous studies have indicated the possibility of human parthenogenetic development. Rhoton-Vlasak et al. in 1996 (13) have shown that short incubations with calcium ionophore can induce pronuclear formation, and recently Nakagawa and collaborators (14) demonstrated that a combination of calcium ionophore and puromycin or DMAP could not only trigger pronucleus formation but early cleavage as well. A similar protocol has also been shown to be applicable in nonhuman primate oocytes (15).

The results disclosed herein show that the present invention provides an effective protocol for parthenogenetic activation of human oocytes, embryonic cleavage, and the formation of a blastocoele cavity. This finding offers the alternative of generating human totipotent stem cells without paternal contribution. **Replace female PN with two male PN (pref having at least one X chromosome)

In addition, the removal of the parthenogenetic female pronucleus and the transfer of two male pronuclei may allow the production of embryos and resulting stem cells for a male donor.

why autologous transplant may still be rejected:

parth-> recomb of DNA may change pattern of gene exp so that transplant triggers immune response

still expect significant reduction in immunorejection, due to HLA matching

Assorted topics to be addressed

Selection of differentiated human donor cell—

Any differentiated cell

Somatic cell or germ cell

Use of senescent/near senescent donor cell to produce rejuvenated cells

Source of oocyte

Cell cycle of donor cell & recipient oocyte

Methods of activation somatic

### EXAMPLES

Human Research Guidelines

Strict guidelines for the conduct of this research have been established by Advanced Cell Technology's independent Ethics Advisory Board (EAB). In order to prevent any possibility of reproductive cloning, the EAB has required careful accounting of all oocytes and embryos used in the research. No embryo created by means of NT technology was maintained beyond 14 days of development. The EAB also established guidelines and oversight for the donor program that provided the human oocytes used in this research. This included extensive efforts to ensure that that the risks to donors were minimized, that donors were fully informed of the risks, and that their consent was free and informed. More information on this subject can be obtained at the Internet website of Advanced Cell Technology. For a review of the ethical issues see (12).

### Example 1

Protocol for reprogramming human somatic cell pronuclei by somatic cell nuclear transfer:

A. Oocyte collection:

1 Oocytes are aspirated from ovarian follicles using an ultrasound-guided needle at 33-34 hrs post hCG administration.

2 Oocytes are denuded of cumulus cells by pipetting up and down using a finely pulled pipette in 1 mg/ml hyaluronidase in Hanks medium.

3 After removing the cumulus cells, the oocytes are placed in Hanks medium with 1% bovine serum albumin (BSA) or with 1% human serum albumin (HSA), and are transported to the laboratory where nuclear transfer procedure is to be performed.

4 Within 1-2 hours after recovery, the oocytes are placed in a drop of 500 μl of G1 (SERIES III) with 5 mg/ml HSA culture medium under mineral oil and are incubated at 37° C. in 6% CO₂ in air until nuclear transfer procedure is performed. Oocytes obtained by this procedure can also be activated to produce a parthenogenetic embryo that can be used for the generation of autologous stem cells (see below).

B. Somatic nuclear donor cell preparation:

1 Non-confluent culture of somatic nuclear donor cells is dissociated and suspended using a solution of trypsin-EDTA in calcium free DPBS for 5 minutes at room temperature. Once a suspension of single cells is obtained, 30% fetal calf serum is added to in order to neutralize the enzymatic activity.

2 The suspension of cells is spun at 500 g for 10 minutes.

3 The supernatant is discarded and the cell pellet is re-suspended with Human Tubule Fluid (HTF; Irvine Scientific, Santa Ana, Calif.) containing 1 mg/ml of HSA. The nuclear donor cells are used in nuclear transfer within 2 hours after dissociation.

Alternatively—

Somatic cells can be taken directly from the donor (e.g. white blood cells or granulosa/cumulus cells from the oocytes) and placed in HTF containing 1 mg/ml of HSA, and are used for nuclear transfer within 2 hours after isolation.

C. Nuclear Transfer:

1 Oocytes are taken from the drop of 500 μl of G1 (SERIES III) with 5 mg/ml HSA culture medium under mineral oil and moved to a drop of 500 μl of G1 (SERIES III) with 5 mg/ml HSA culture medium a containing 1 μg/ml 33342 Hoechst dye, and are incubated for 15 minutes under mineral oil at 37° C. in 6% CO₂ in air.

2 Somatic nuclear donor cells are placed into a manipulation drop of 100 μl of HTF containing 1 mg/ml of HSA, 20% FCS and 10 μg/ml of cytochalasin B under mineral oil.

3 Oocytes are moved into a manipulation drop of 100 μl of HTF containing 1 mg/ml of HSA, 20% FCS and 10 μg/ml of cytochalasin B under mineral oil, adjacent to the drop containing the somatic cells, and the whole plate (100 mm Falcon) is placed at 37° C. in the warming stage of the microscope.

4 After 10 minutes of incubation, the oocyte's metaphase II plate is visualized using an ultraviolet light for no more than 5 seconds; and a laser (______) is used to drill a 20 micron hole in the zona pellucida adjacent to the oocyte's metaphase II plate.

5 The oocyte chromosomes are removed by suction into a fire-polished 20 μm I.D. glass pipette without compromising the integrity of the oocyte.

6 One small somatic cell is picked up using a fire-polished 20 μm I.D. glass pipette and is placed in the perivitelline space of the oocyte.

7 Couplets (oocyte and somatic cell) are moved from the manipulation drop to into a drop of 500 μl of G1 (SERIES III) with 5 mg/ml HSA culture medium under mineral oil, and are incubated at 37° C. in 6% CO₂ in air until fusion is performed.

8 Fifteen minutes after cell transfer, couplets are moved out of the drop of 500 μl of G1 (SERIES III) with 5 mg/ml HSA culture media into a 30 mm Falcon plate containing 3 ml of HTF with 1 mg/ml of HSA for 30 seconds.

9 Couplets are moved to a solution of 50% HTF with 1 mg/ml of HSA and 50% fusion media (Sorbitol based) for 1 minute.

10 Couplets are moved to a solution of 100% Sorbitol fusion medium.

11 Couplets are moved to a BTX fusion chamber (500 μl gap) filled with Sorbitol fusion media and placed between two electrodes.

12 Alignment of the couplets is performed manually using a glass pipette in a way that the axis of the somatic cell and oocyte is perpendicular to the axis of the electrodes.

13 A fusion pulse of 150 volts for 15 μseconds is delivered.

14 Couplets are immediately moved into a solution of 50% HTF with 1 mg/ml of HSA and 50% Sorbitol fusion medium for 1 minute.

15 Couplets are moved into a 30 mm Falcon plate containing 3 ml of HTF with 1 mg/ml of HSA for 1 minute.

16 Couplets are moved into the incubator into a drop of 500 μl of G1 (SERIES III) with 5 mg/ml HSA culture media under mineral oil at 37° C. in 6% CO₂ in air until activation is performed.

D. Activation

1 At 45 hours after hCG administration, fused reconstructed embryos are placed into a solution of 10 μM ionomycin in HTF with 1 mg/ml of HSA for 5 minutes.

2 Reconstructed embryos are moved into a drop of 500 μl of a solution of 2 mM of 6-DMAP in G1 (SERIES III) with 5 mg/ml HSA culture media under mineral oil 37° C. in 6% CO₂ in air for 4 hours

1 Reconstructed embryos are taken out of DMAP solution and rinsed three times in three different 30 mm plates of HTF with 1 mg/ml of HSA

2 Reconstructed embryos are moved into a drop of 500 μl of G1 (SERIES III) with 5 mgr/ml HSA culture media under mineral oil at 37° C. in 6% CO₂ in air

E. Culturing the reconstructed embryos:

1 For the first 72 hours, reconstructed embryos are cultured in a drop of 500 μl of G1 (SERIES III) with 5 mg/ml HSA culture media under mineral oil, at 37° C. in 6% CO₂ in air.

2 For the rest of the culture period (from hour 73 until blastocyst), the embryos are cultured in a drop of 500 μl of KSOM+AA+Glucose (Specialty media) with 5 mg/ml HSA and 10% heat inactivated follicular fluid obtained from superovulated human oocyte donors, under mineral oil at 37° C. in 6% CO₂ in air.

F. Inner Cell Mass Isolation:

Once blastocysts are generated, the inner cell mass (ICM) can be isolated.

1 Hatched blastocysts are placed in tyroid acid for a few seconds until the zona pellucida is digested and subsequently moved to HTF with 1 mg/ml of HSA for 2 minutes.

2 Blastocysts are moved to solution of polyclonal antibodies (1:5) of serum against BeWo cells in G1 (SERIES III) without HSA for one hour.

3 Embryos are rinsed 3 times in HTF with 1 mg/ml of HSA, and are moved to a solution of guinea pig complement (1:3) in G1 (SERIES III) without HSA until trophoblast lysis occurs.

4 ICM is rinsed in HTF with 1 mg/ml of HSA. The ICM is then placed on a layer of mitotically inactivated mouse embryonic fibroblasts in DMEM with 15% fetal calf serum and is cultured to generate embryonic stem cells.

### Example 2

Superovulation and oocyte retrieval:

Oocyte donors were 12 women between the ages of 24 and 32 years with at least one biologic child. They underwent thorough psychological and physical examination, including assessment by the Minnesota Multiphasic Personality Index test, hormone profiling, and PAP screening. They were also screened carefully for infectious diseases, including hepatitis viruses B and C, human immunodeficiency virus, and human T-cell leukemia virus. Donor ovaries were down-regulated by at least 2 weeks of oral contraceptives, followed by controlled ovarian hyperstimulation with twice daily injections of 75-150 units of gonadotropins. Pituitary suppression was maintained in some donors by concomitant twice daily administration of Synarel, beginning 3 days before discontinuing oral contraceptives and 5 days before initiating gonadotropin injections, and in other donors by injection of Antigone beginning with leading follicle diameters of 12 mm. Ovarian stimulation was calculated to minimize the risk of ovarian hyperstimulation syndrome by ensuring the serum estradiol levels of the donor did not exceed 3,500 μg/ml on the day of human chorionic gonadotropin (hCG) injection to stimulate the resumption of oocyte meiosis. Blood serum estradiof levels were measured at least every 2 days, and hCG was administered when the leading follicle reached at least 18 mm by ultrasound examination. Oocytes were collected from antral follicles of anesthetized donors by ultrasound-guided needle aspiration into sterile test tubes. They were freed of cumulus cells with hyaluronidase and scored for stage of meiosis by direct examination.

Oocyte maturation profile

A total of 71 oocytes were obtained from seven volunteers (Table 1). At the time of retrieval, five oocytes were at the germinal vesicle stage, and no further development was observed after 48 h in culture. Nine oocytes were at metaphase I (MI) stage and were systematically used for activation or NT after ˜3 h culture. Fifty-seven oocytes that were at metaphase II (MII) stage were immediately used for NT or parthenogenetic activation experiments.

TABLE 1
Maturation profile of Human Oocytes at the Time of Collection

| | No. of | Germinal | | |
|---|---|---|---|---|
| Donor | oocytes | vesicle | MI | MII |
| 1 | 6 | 1 | 0 | 5 |
| 2 | 15 | 0 | 0 | 15 |
| 3 | 8 | 2 | 0 | 6 |
| 4 | 11 | 2 | 4 | 5 |
| 5 | 15 | 0 | 2 | 13 |
| 6 | 11 | 0 | 3 | 8 |
| 7 | 5 | 0 | 0 | 5 |
| Total | 71 | 5 | 9 | 57 |

### Example 3

Reprogramming human somatic cell nuclei/chromatin in embryos reconstituted by nuclear transfer:

A. Somatic cell isolation

Adult human fibroblasts were isolated from 3-mm skin biopsies for use as somatic nuclear donor cells. The people from who the skin biopsies were taken from consenting adult volunteers of varying ages who were generally healthy, or who had a disorder such as diabetes or spinal cord injury that might benefit from therapeutic transplantation of autologous cells produced by cloning by nuclear transfer. Skin explants were cultured for 3 weeks in DMEM (Gibco, Grand Island. N.Y.) plus 10% fetal calf serum (HyClone, Logan, Utah) at 37° C. and 5% CO₂. Once cellular outgrowth was observed, fibroblasts and keratinocytes were enzymatically dissociated using 0.25% trypsin and 1 mM EDTA (GibcoBRL, Grand Island, N.Y.) in PBS (GibcoBRL) and passaged 1:2. Fibroblasts were used at the second passage. The identity of these cells was later confirmed by immunocytochemistry, and seed stocks of these cells were frozen and stored in liquid nitrogen until use as cell donors.

Cumulus cells were used immediately after oocyte retrieval and processed as previously described (11). The cumulus-oocyte complexes were treated in HEPES-CZB medium (Chatot et al., 1989, J. Reprod. Fertil. 86:679-688) with 1 mg/ml hyaluronidase to disperse the cumulus cells. Following dispersal, the cumulus cells were transferred to HEPES-CZB medium containing 12% (w/v) PVP, and were kept at room temperature for up to 3 hours before injection.

B. Oocyte enucleation and nuclear transfer:

Prior to manipulation, oocytes were incubated with 1 μg/ml bisbenzimide (Sigma, St. Louis, Mo.) and cytochalasin B (5 ng/ml; Sigma) in embryo culture medium for 20 min. All manipulations were made in HEPES-buffered HTF under oil. Chromosomes were visualized with a 200X power on an inverted microscope equipped with Hoffman optic and epifluorescent ultraviolet light. Enucleation was performed using a piezo electric device (Prime Tech, Japan) specially designed to minimize the damage generated during the micromanipulation procedure. A 10 μm I.D. blunt needle that contained mercury near its tip to be able to control the penetration capacity and accuracy of the procedure was used to penetrate gently the zona pellucida and aspirate the chromosomes and adjacent cytosol. Nuclear donor cells were maintained in a solution of 12% polyvinylpyrrolidone (PVP, Irvine Scientific) in culture media and loaded into a small piezo-driven needle of approximately 5 μm I.D. Donor nuclei were isolated from fibroblast cells by suctioning the cells in and out through the pipette. Each isolated fibroblast nucleus was immediately injected into the cytosol of an enucleated oocyte. Cumulus cells are half the size of fibroblasts, and each cumulus cell was injected as a whole cell into an enucleated oocyte. After nuclear transfer, the reconstructed cells were returned to the incubator, and were activated one to three hours later.

C. Activation and culture of the reconstructed oocytes.

At 35-45 hours after exogenous hCG stimulation, oocytes were activated by incubating them with 5 μM ionomycin (Calbiochem, La Jolla, Calif.) for 4 min, followed by 2 mM 6-dimethylaminopurine (DMAP; Sigma) in G1.2 for 3 h. The oocytes were then rinsed three times in HTF and placed in G1.2 (Vitrolife, Vero Beach, Fla.) or in Cook-Cleavage culture medium (Cook IVF, Indianapolis, Ind.) for 72 h at 37° C. in 5% CO₂. On the fourth day of culture, cleaving oocytes resembling embryos were moved to G2.2 or Cook-Blastocyst culture medium until day 7 after activation.

D. Nuclear transfer and reprogramming of donor cell nuclei

Oocytes from seven volunteers were used for nuclear transfer procedures. A total of 19 oocytes were reconstructed using nuclei from fibroblasts and cumulus cells. Twelve hours after reconstruction with a fibroblast nucleus, seven oocytes (69%, as a percentage of reconstructed oocytes) exhibited a single, large pronucleus, morphologically similar to those observed in oocytes fertilized with sperm. Only one pronucleus with prominent nucleoli (up to 10) was observed in each reconstructed oocyte. None of the embryos reconstructed with fibroblast nuclei in this round of experiments underwent cleavage. Four of eight oocytes injected with cumulus cells developed pronuclei, and three of those cleaved to four or six cells. The results of these nuclear transfer procedures are summarized in Table 2.

TABLE 2
Somatic Cell Nuclear Transfer in Human Oocytes

| | | Reconstructed | Pronucleus | Cleaved |
|---|---|---|---|---|
| Donor | Cell type | oocytes | (%)^{a} | (%)^{b} |
| 3 | Fibroblast | 2 | 0 | 0 |
| 4 | | 5 | 4 (80) | 0 |
| 5 | | 4 | 3 (75) | 0 |
| 6 | Cumulus | 5 | 3 (60) | 3 (100) |
| 7 | | 3 | 1 (33) | 0 |
| Total | | 19 | 11 (58) | 3 (27) |

^{a}As a percentage of reconstructed oocytes.
^{b}As percentage of pronuclear embryos.

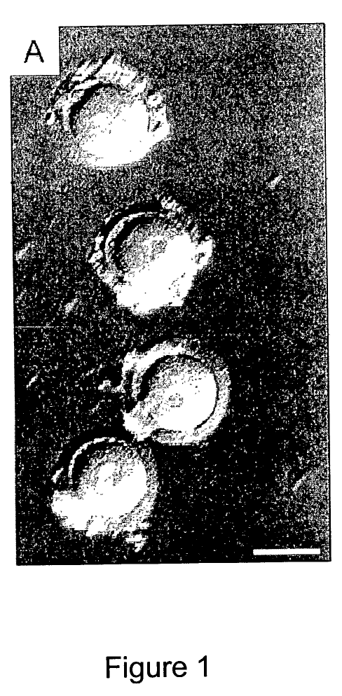

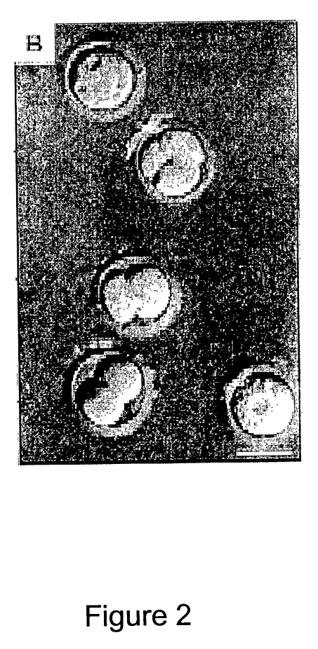

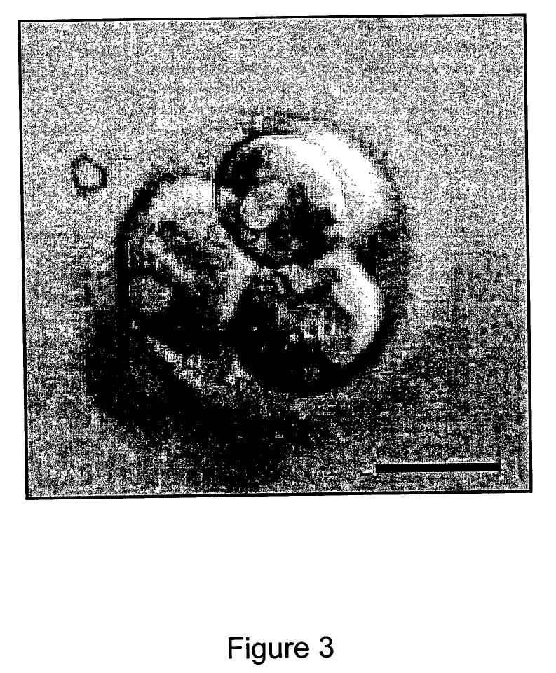

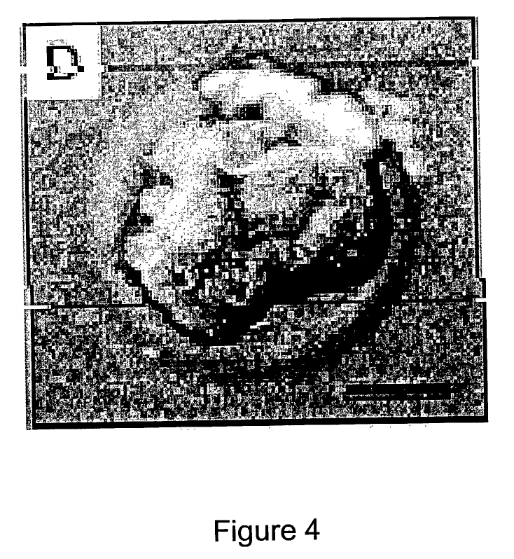

FIGS. 1-4 show cleavage-stage embryos derived from reconstructed oocytes produced by nuclear transfer using cumulus cells as the nuclear donor cells. FIGS. 1 and 2 show pronuclear-stage embryos at 12 h and 36 h, respectively. The scale bars=100 μm. FIGS. 3 and 4 show a four-cell embryo and a six-cell embryo, respectively, at 72 h. The nuclei of the embryos were stained with bisbenzimide (Sigma) and visualized under UV light. The scale bars=50 μm.

These results demonstrate production of embryonic pronuclei following nuclear transfer using two different cell types: adult cumulus cells and skin fibroblasts. Using cumulus cells as donors, three oocytes cleaved to the two-cell, four-cell, and six-cell stages, respectively. Oocytes reconstituted with cultured adult fibroblasts developed pronuclei but did not cleave.

E. Cleavage by oocytes reconstituted with fibroblast nuclei

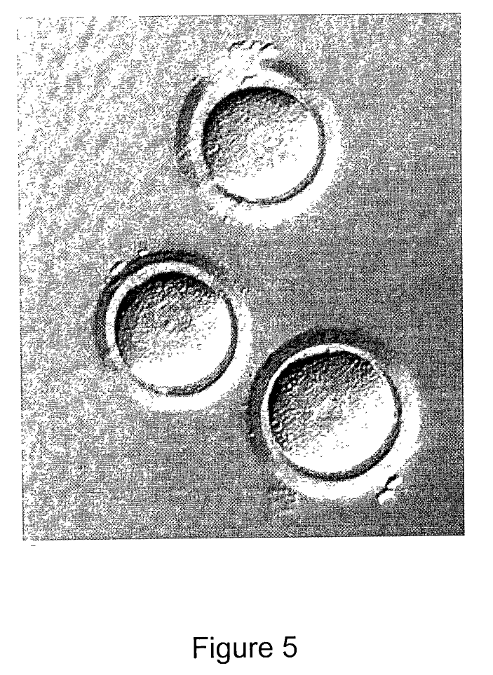

In a subsequent study similar to the one described above, the nuclei of two human dermal fibroblasts were transferred into enucleated human oocytes using the above-described methods, and one of the reconstituted embryos underwent cleavage to produce the cleavage-stage embryo shown in FIG. 5.

### Example 4

Production of Autologous Cells by Parthenogenetic Activation of Oocytes

Oocytes from three volunteers were used for parthenogenetic activation. The donors were induced to superovulate by 11 days of low dose (75 IU bid) gonadotropin injections prior to hCG injection. A total of 22 oocytes were obtained from the donors 34 hours after HCG stimulation, and were activated at 40-43 h after hCG stimulation.

The oocytes were activated on day 0, using the ionomycin/DMAP activation protocol described above. Twelve hours after activation, 20 oocytes (90%) developed one pronucleus and cleaved to the two-cell to four-cell stage on day 2. On day 5 of culture, evident blastocoele cavities were observed in six of the parthenotes (30% of the cleaved oocytes) though none of the embryos displayed a clearly discernible inner cell mass. The results of parthenogenetic activation of the human oocytes are summarized in Table 3.

TABLE 3
Parthenogenetic Activation of Human Oocytes

| | | | | Embryos with |
|---|---|---|---|---|
| | No. of | | | blastocoele |
| Donor | oocytes | Pronucleus (%)^{a} | Cleaved (%)^{a} | cavity^{b} |
| 1 | 5 | 4 (80) | 4 (80) | 0 |
| 2 | 14 | 13 (93) | 13 (93) | 4 (31) |
| 6 | 3 | 3 (100) | 3 (100) | 2 (67) |
| Total | 22 | 20 (90) | 20 (90) | 6 (30) |

^{a}As a percentage of activated oocytes.
^{b}As percentage of cleaved oocytes.

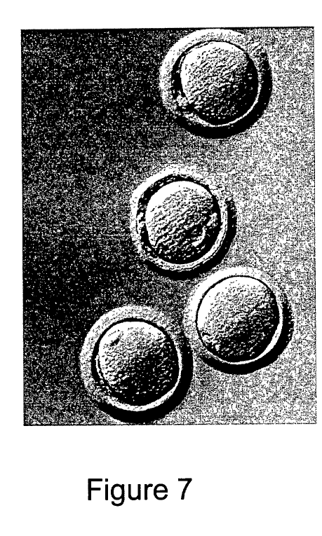

FIGS. 7-10 show embryos and stem cells produced by parthenogenetic activation of human oocytes. FIG. 7 shows MII oocytes at the time of retrieval. embryos underwent cleavage to produce the cleavage-stage embryo shown in FIG. 5.

### Example 4

Production of Autologous Cells by Parthenogenetic Activation of Oocytes

Oocytes from three volunteers were used for parthenogenetic activation. The donors were induced to superovulate by 11 days of low dose (75 IU bid) gonadotropin injections prior to hCG injection. A total of 22 oocytes were obtained from the donors 34 hours after HCG stimulation, and were activated at 40-43 h after hCG stimulation.

The oocytes were activated on day 0, using the ionomycin/DMAP activation protocol described above. Twelve hours after activation, 20 oocytes (90%) developed one pronucleus and cleaved to the two-cell to four-cell stage on day 2. On day 5 of culture, evident blastocoele cavities were observed in six of the parthenotes (30% of the cleaved oocytes) though none of the embryos displayed a clearly discernible inner cell mass. The results of parthenogenetic activation of the human oocytes are summarized in Table 3.

TABLE 3
Parthenogenetic Activation of Human Oocytes

| | | | | Embryos with |
|---|---|---|---|---|
| | | | | blastocoele |
| | No. of | | | cavity |
| Donor | oocytes | Pronucleus (%)^{a} | Cleaved (%)^{a} | (%)^{b} |
| 1 | 5 | 4 (80) | 4 (80) | 0 |
| 2 | 14 | 13 (93) | 13 (93) | 4 (31) |
| 6 | 3 | 3 (100) | 3 (100) | 2 (67) |
| Total | 22 | 20 (90) | 20 (90) | 6 (30) |

^{a}As a percentage of activated oocytes.
^{b}As percentage of cleaved oocytes.

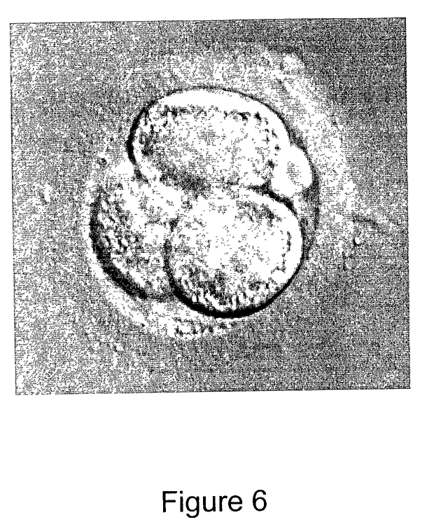

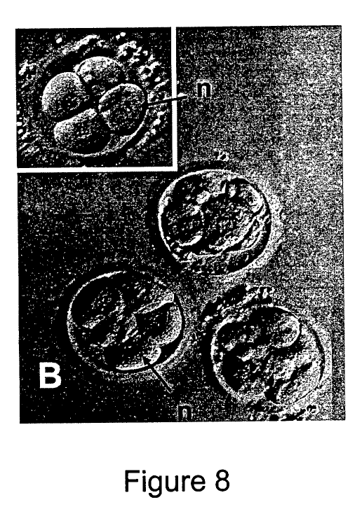

FIG. 6 shows MII oocytes at the time of retrieval. FIG. 7 shows four-to six-cell embryos 48 h after activation. Distinguishable single-nucleated blastomeres (labeled “n” in FIG. 6) were consistently observed. FIG. 8 shows embryos with blastocoele cavities (arrows) that were detected on day 6 and maintained in culture until day 7. The scale bars for FIGS. 6-8=100 μm.

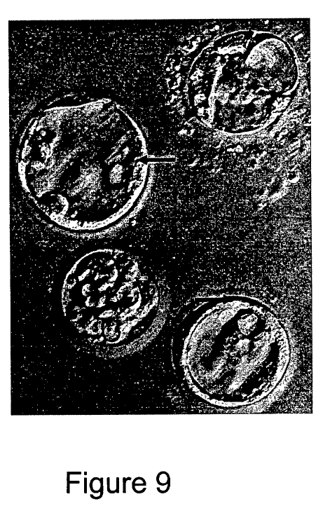

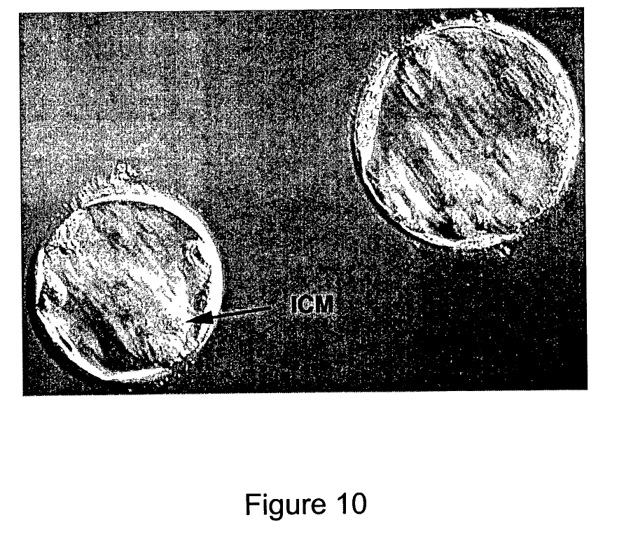

In a study similar to the one described above, human oocytes were activated using the ionomycin/DMAP activation protocol and were cultured in vitro. One of the activated embryos developed a pronucleus, cleaved, formed a blastocoele cavity, and then developed into a blastocyst having an inner cell mass, shown in FIG. 9. The inner cell mass was isolated and plated on mouse feeder layers as described (Cibelli, J. B., et al. 2002. Parthenogenetic stem cells in nonhuman primates. Science 295: 819). The cultured ICM cells increased in number over the first week, and cells indistinguishable from human embryonic stem cells were observed. These grew in close association as a colony with a distinct boundary, as shown in FIG. 10; they had a high nuclear-to-cytoplasmic ratio, prominent nucleoli, and were observed to differentiate in vitro into multiple differentiated cell types.

### REFERENCES

1. Lanza RP, Cibelli JB, and West MD. (1999) Prospects for the use of nuclear transfer in human transplantation. Nat Biotechnol 17:1171-1174.

2. Brown JP, Wei W, and Sedivy JM. (1997) Bypass of senescence after disruption of p21CIP1/WAF1 gene in normal diploid human fibroblasts. Science 277:831-833.

3. National Academy of Sciences. (2001) Stem Cells and the Future of Regenerative Medicine. National Academy Press, Washington, D.C.

4. Collas P, and Barnes FL. (1994) Nuclear transplantation by microinjection of inner cell mass and granulosa cell nuclei. Mol Reprod Dev 38:264-267.

5. Wilmut I, Schnieke AE, McWhir J, Kind AJ, and Campbell KHS. (1997) Viable offspring derived from fetal and adult mammalian cells. Nature 385:810-813.

6. Cibelli JB, Stice SL, Golueke PJ, Kane JJ, Jerry J, Blackwell C, Ponce de Leon FA, and Robl JM. (1998) Cloned transgenic calves produced from nonquiescent fetal fibroblasts. Science 280:1256-1258.

7. Munsie MJ, Michalska AE, O'Brien CM, Trounson AO, Pera MF, and Mountford PS. (2000) Isolation of pluripotent embryonic stem cells from reprogrammed adult mouse somatic cell nuclei. Curr Biol 10:989-992.

8. Kawase EYY, Yagi T, Yanagimachi R, and Pedersen RA. (2000) Mouse embryonic stem (ES) cell lines established from neuronal cell-derived cloned blastocysts. Genesis 28:156-163.

9. Wakayama T, Tabar V, Rodriguez I, Perry AC, Studer L, and Mombaerts P. (2001) Differentiation of embryonic stem cell lines generated from adult somatic cells by nuclear transfer. Science 292:740-743.

10. Cibelli JB, Stice SL, Golueke PJ, Kane JJ, Jerry J, Blackwell C, Ponce de Leon FA, and Robl JM. (1998) Transgenic bovine chimeric offspring produced from somatic cell-derived stem-like cells. Nat Biotechnol 16:642-646.

11. Wakayama T, Perry AC, Zuccotti M, Johnson KR, and Yanagimachi R. (1998) Full-term development of mice from enucleated oocytes injected with cumulus cell nuclei. Nature 394:369-374.

12. Lanza RP, Caplan AL, Silver LM, Cibelli JB, West MD, and Green RM. (2000) The ethical validity of using nuclear transfer in human transplantation. JAMA 284:3175-3179.

13. Rhoton-Vlasak A, Lu PY, Barud KM, Dewald GW, and Hammitt DG. (1996) Efficacy of calcium ionophore A23187 oocyte activation for generating parthenotes for human embryo research. J Assist Reprod Genet 13:793-796.

14. Nakagawa K, Yamano S, Nakasaka H, Hinokio K, Yoshizawa M, and Aono T. (2001) A combination of calcium ionophore and puromycin effectively produces human parthenogenones with one haploid pronucleus. Zygote 9:83-88.

15. Mitalipov SM, Nusser KD, and Wolf DP. (2001) Parthenogenetic activation of rhesus monkey oocytes and reconstructed embryos. Biol Reprod 65:253-259.

16. Cheong HT, Takahashi Y, and Kanagawa H. (1994) Relationship between nuclear remodeling and subsequent development of mouse embryonic nuclei transferred to enucleated oocytes. Mol Reprod Dev 37:138-145.

17. Dominko TR-SJ, Chan A, Moreno RD, Luetjens CM, Hewitson L, Takahashi D, Martinovich C, White JM, and Schatten G. (1999) Optimization strategies for production of mammalian embryos by nuclear transfer. Cloning 1:143-152.

18. Bondioli KR. (1993) Nuclear transfer in cattle. Mol Reprod Dev 36:274-275.

19. Campbell KHS, Ritchie WA, and Wilmut I. (1993) Nuclear-cytoplasmic interactions during the first cell cycle of nuclear transfer reconstructed bovine embryos: implications for deoxyribonucleic acid replication and development. Biol Reprod 49:933-942.

20. Ohgane J, Wakayama T, Kogo Y, Senda S, Hattori N, Tanaka S, Yanagimachi R, and Shiota K. (2001) DNA methylation variation in cloned mice. Genesis 30:45-50.

21. Tao T, Machaty Z, Boquest AC, Day BN, and Prather RS. (1999) Development of pig embryos reconstructed by microinjection of cultured fetal fibroblast cells into in vitro matured oocytes. Anim Reprod Sci 56:133-141.

22. Terlouw SL, Stumpf TT, Funahashi H, Prather RS, and Day BN. (1993) Pig oocyte activation and processing of transplanted nuclei. Theriogenology 39:329.

23. Wakayama T, and Yanagimachi R. (2001) Mouse cloning with nucleus donor cells of different ages and type. Mol Reprod Dev 58:376-383.

24. Klug MG, Soonpaa MH, Koh GY, and Field LJ. (1996) Genetically selected cardiomyocytes from differentiating embryonic stem cells form stable intracardiac grafts. J Clin Invest 98:216-224.

25. Brustle O, Jones KN, Learish RD, Karram K, Choudhary K, Wiestler OD, Duncan ID, and McKay RD. (1999) Embryonic stem cell-derived glial precursors: a source of myelinating transplants. Science 285:754-756.

26. McDonald JW, Liu XZ, Qu Y, Liu S, Mickey SK, Turetsky D, Gottlieb Dl, and Choi DW. (1999) Transplanted embryonic stem cells survive, differentiate and promote recovery in injured rat spinal cord. Nat Med 5:1410-1412.

27. Soria B, Roche E, Berna G, Leon-Quinto T, Reig JA, and Martin F. (2000) Insulin-secreting cells derived from embryonic stem cells normalize glycemia in streptozotocin-induced diabetic mice. Diabetes 49:157-162.

28. Lanza RP, Langer R, and Vacanti JP. (2000) Principles of Tissue Engineering. Academic Press, San Diego.

29. Atala A, and Lanza RP. (2001) Methods of Tissue Engineering.

Academic Press, San Diego.

We claim:

1. A method for producing a diploid human pronudeus comprising exposing the nucleus of a differentiated human cell to cytoplasm of an oocyte.
